(19) United States
(12) Patent Application Publication
(10) Pub. No.: US 2015/0005084 A1
(43) Pub. Date: Jan. 1, 2015

(54) TWO-ENVIRONMENT GAME PLAY SYSTEM

(71) Applicant: Origin, LLC, Okahoma City, OK (US)

(72) Inventors: Ryan S. Tawwater, Oklahoma City, OK (US); John M. Vollbrecht, Oklahoma City, OK (US); James T. Vollbrecht, Oklahoma City, OK (US)

(73) Assignee: ORIGIN, LLC, Oklahoma City, OK (US)

(21) Appl. No.: 14/321,333

(22) Filed: Jul. 1, 2014

Related U.S. Application Data
(60) Provisional application No. 61/841,544, filed on Jul. 1, 2013.

(51) Int. Cl. A63B 24/00; A63B 69/36

(52) U.S. Cl. CPC A63B 24/0021; A63B 24/0003; A63B 69/3691; A63B 2024/0031; A63B 2220/803; A63B 2220/05
USPC 473/156

(57) ABSTRACT

A game-play environment includes a tee box, a range surface, and a monitor. The tee box is configured to permit a player to hit a golf ball onto the range surface. The range surface has a plurality of physical markers. The monitor depicts a virtual environment that includes a plurality of virtual components. Some of the virtual components are visual cues that correspond to the physical markers. A player is able to play the game by targeting the appropriate physical marker that corresponds to the desired visual cue.

### CROSS-REFERENCE TO RELATED APPLICATIONS

This application claims the benefit of U.S. Application No. 61/841,544 for Two-Environment Game Play System.

This invention relates to a system and method related to a game play environment.

### BACKGROUND OF THE INVENTION

Conventional methods of tracking an object (e.g. golf ball, basketball, baseball, etc.) employ various types of sensors including Doppler radar technology, camera based technology, high speed 3D camera based technology, and stereoscopic sensors. The sensors can be configured to track the object and with the aid of a computer can recreate the movement of the object in a computerized virtual environment. In athletic application, these tracking systems have been used to provide feedback for coaching, player development, and other training/improvement applications, with focus on the movement of a virtual object relative to a virtual environment. The prior art is principally focused on providing analysis about the player's technique and the resulting effect on the flight path of the object. These systems have found a particular benefit in the area of golf instruction.

Conventional indoor golf simulators utilize sensors, as mentioned above, and represent data points in an indoor virtual space. Such simulators monitor the ball with sensors, which capture the XYZ data points and relay those data points to a computer system that creates a representation of the data points in a virtual space, such as a virtualized hole on a golf course. The prior art focuses on capturing the data points and incorporating the data points into a purely virtual environment, with no identifiable links to the physical environment where the golf ball was actually hit.

It is apparent that there is a need for a system and method of tracking a ball, or other object, and rendering the flight path of that ball in a virtual gaming environment that is coordinated with the physical environment in which the ball is struck. The present invention is focused on solving such a need and providing the techniques thereby to fulfill that need.

### BRIEF SUMMARY OF THE INVENTION

Described herein is a game-play environment that includes a tee box, a range surface, and a monitor. The tee box is configured to allow a player to hit a golf ball onto the range surface. The range surface has a plurality of physical markers. The monitor is positioned so that the player can see the monitor while in the tee box. The monitor depicts a virtual environment that corresponds to a desired virtual game. Depending on the particular game selected, a set of virtual components are displayed on the monitor. Some of these virtual components are visual cues that correspond to the physical markers on the range surface. The player can achieve the game's objectives by targeting the appropriate physical marker that corresponds to the desired visual cue.

### BRIEF SUMMARY OF THE SEVERAL VIEWS OF THE DRAWING

FIG. 1 depicts a perspective view of a physical environment that is used in the game play.

FIG. 2 depicts a side perspective view of a virtual environment showing the path a ball travels through that virtual environment.

FIG. 3 depicts a front perspective view of a first preferred embodiment of a virtual environment.

FIG. 4a depicts a top view of a second preferred embodiment of a virtual environment.

FIG. 4b depicts a top view of the second preferred embodiment from FIG. 4a wherein the virtual environment has been adjusted for aiming.

FIG. 4c depicts a top view of the second preferred embodiment from FIGS. 4a & 4b wherein the virtual environment has been further adjusted for aiming.

### DETAILED DESCRIPTION OF THE INVENTION

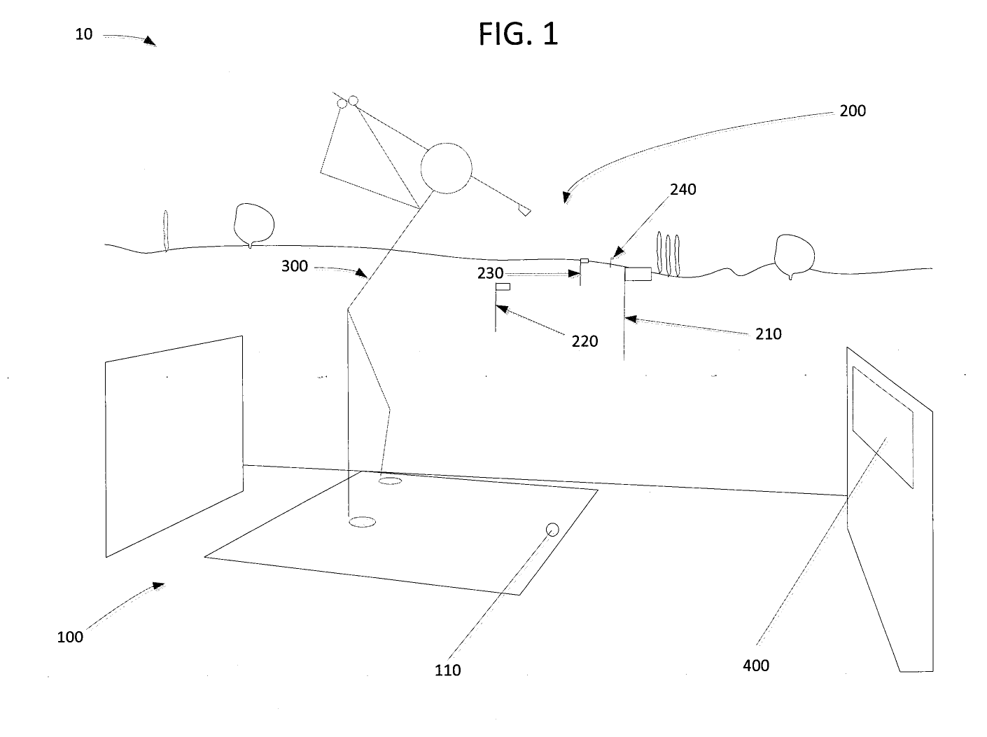

In accordance with a preferred embodiment of the present invention, a game-play environment 10 includes a tee box 100 and a range surface 200. The tee box 100 includes a ball 110 and a monitor 400. The range surface 200 includes a plurality of physical markers 210, 220, 230, and 240. FIG. 1 is a perspective view of a preferred embodiment of the game-play environment 10. Shown therein is a player 300 positioned in the tee box 100 over the golf ball 110 and facing the monitor 400. It is understood that the particular position of the player 300 within the tee box 100 enables that player 300 to see the physical markers 210, 220, 230, and 240 and also to see the monitor 400. It will be further understood that while FIG. 1 depicts a preferred embodiment of the game-play environment 10, other embodiments and arrangements of the constituent parts of the game-play environment 10 are possible. For example, the monitor 400 can be raised or mounted on a standard pivot mount for adjustable positioning within the tee box 100. Similarly, the positioning of the plurality of physical markers on the range surface 200 can be adjusted as desired for a particular embodiment. It is preferred, however, that the plurality of physical markers generally be distributed throughout the range surface 200 to facilitate game play, as discussed below. In the preferred embodiment, the physical markers are distributed throughout the range surface 200 at distances of approximately 60 yards, 130 yards, 180 yards, and 210 yards from the tee box 100.

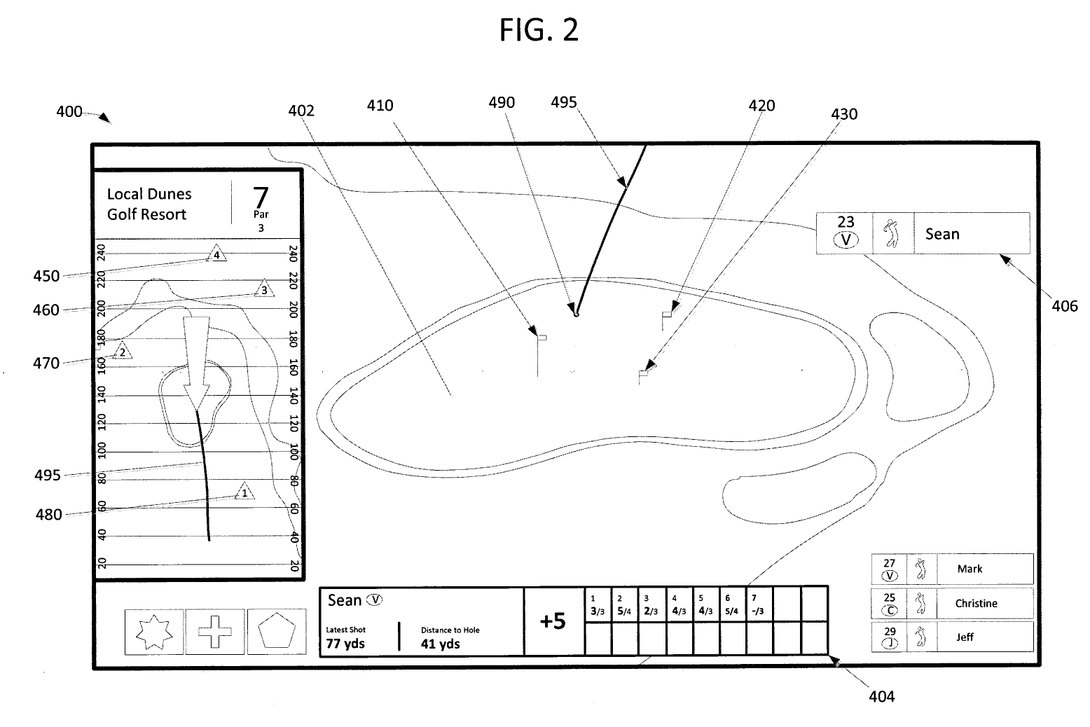

Turning to FIG. 2, depicted therein is a virtual environment that is displayed to the player 300 on the monitor 400. In the preferred embodiment, the virtual environment 10 is configured to play a virtual game of golf. Accordingly, the virtual environment shown in FIG. 2 consists of a plurality of visual components appropriate for a golf game, including without limitation, a golf green 402, a player score card 404, a current player information box 406, a plurality of player location flags 410, 420, and 430, a plurality of visual cues 450, 460, 470, and 480, a virtual golf ball 490, and a golf ball flight path 495.

Some of these visual components correspond with physical aspects of the range surface 200 and tee box 100. For example, the visual cues 450, 460, 470, and 480 correspond with the physical markers 210, 220, 230, and 240 respectively. Importantly, the relative positions and distances between the physical markers 210, 220, 230, and 240 are the same relative positions and distances depicted between the visual cues 450, 460, 470, and 480. It will be understood that by depicting a plurality of visual cues in the virtual environment that correspond to a plurality of physical markers on the range surface 200, various desirable features of the golf game become possible. It will be further understood that other games can benefit from the correspondence of physical markers with visual cues, including without limitation baseball, football, ultimate frisbee, tennis, and others.

One such benefit is that after a player 300 strikes the golf ball 110, the place that the golf ball 110 comes to rest on the range surface 200 can be depicted within the virtual environment as being in a position and distance from each of the plurality of visual cues that corresponds to position and distance of the golf ball 110 from each of the plurality of physical markers on the range surface. For example, if the resting place of the golf ball 110 is 10 feet north of physical marker 220, 15 feet west of physical marker 230, and 40 feet south of physical marker 240, the monitor 400 will display a virtual golf ball 490 as being 10 feet north of visual cue 450, 15 feet west of visual cue 460, and 40 feet south of 470.

Another benefit of depicting a plurality of visual cues in the virtual environment that correspond to a plurality of physical markers on the range surface 200, is that the actual path that the golf ball 110 travels from the tee box 110 to the range surface 200 can be depicted within the virtual environment and displayed on the monitor 400. FIG. 2 depicts this path 495.

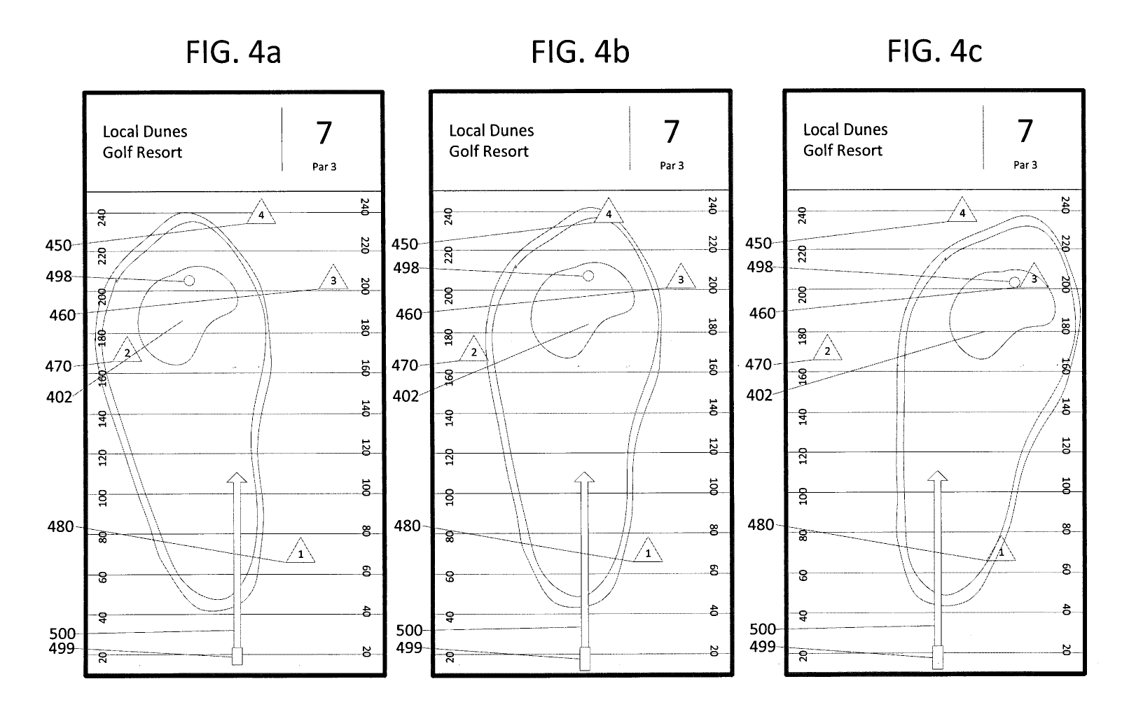

Yet another benefit of depicting a plurality of visual cues in the virtual environment that correspond to a plurality of physical markers on the range surface 200 is that the player 300 can use the plurality of physical markers as targets that correspond to particular visual components depicted within the virtual environment. For example turning to FIG. 4a, if in the particular game being played in the game-play environment 10 it is desirable to get the virtual golf ball 490 to rest near visual component 498 (which his depicted as the cup on a golf hole), then the player 300 can adjust his golf shot (by changing golf clubs, stroke mechanics, foot position, body position, etc.) to improve the chances that the golf ball 110 will come to rest near physical marker 210, which corresponds to visual cue 450, the visual cue closest to visual component 220.

It will be understood that the virtual environment may also be adjusted so that the visual cues 450, 460, 470, and 480 that correspond to physical markers 210, 220, 230, and 240 are better aligned with the desired visual components. For example, FIG. 4b depicts the same virtual environment of FIG. 4a, except that the perspective has been selectively rotated so that visual cue 460 is now in closer proximity to visual component 498. FIG. 4c shows that the perspective can be adjusted even further bring in visual cue 460 is even closer proximity to visual component 498. Thus, the player 300 may now improve their chances of getting the virtual golf boll 490 to rest near visual component 498 by aiming their golf shot at physical marker 220, which corresponds to visual cue 460.

It will be further understood that other visual components can be displayed to assist the player 300 in aiming. For example, in FIGS. 4a, 4b and 4c, a virtual tee box 499 is shown, which corresponds to the tee box 100, and a directional indicator 500 is displayed to indicate in what direction the virtual golf ball 490 will travel if the player 300 hits the golf ball 110 on a heading of 0 degrees (directly straight down the range surface 200).

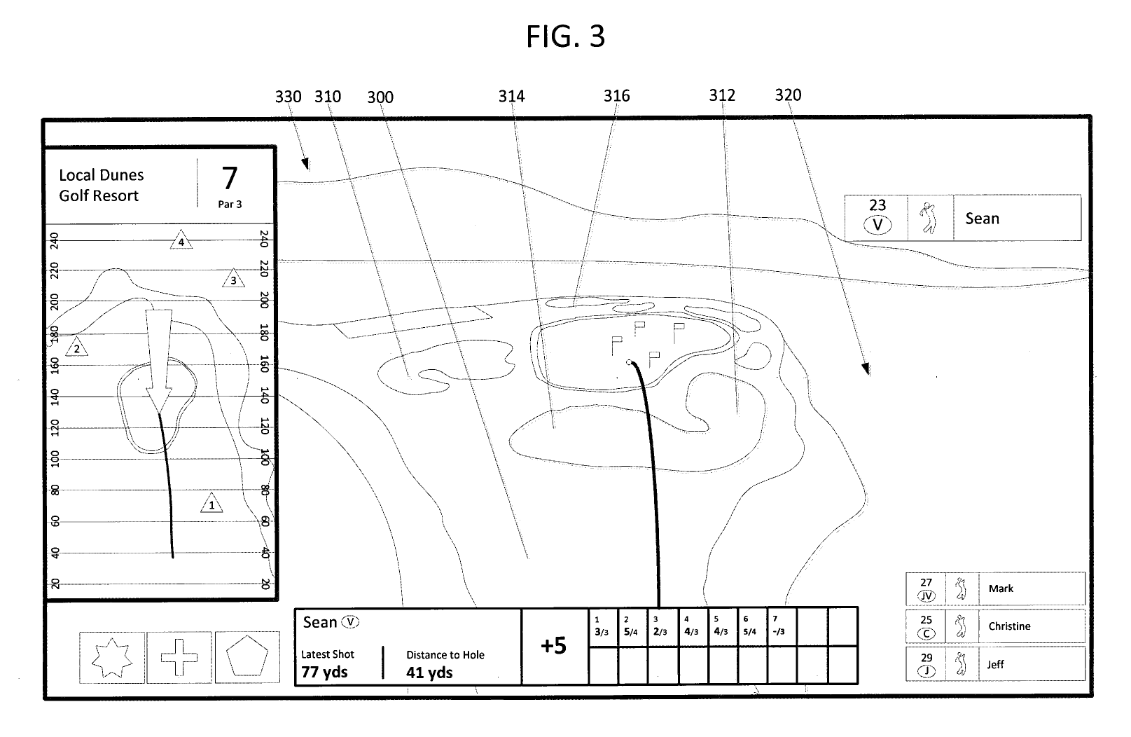

It will be understood that other visual components appropriate to a golf game, if desired, may be used in the preferred embodiment, including without limitation fairways, sand traps, virtual tee boxes, water hazards, and out of bounds markers. In addition, it may be desirable to include other visual components to be depicted within the virtual environment that do not necessarily affect the play of the virtual golf game, but rather fill the background of the virtual environment, including without limitation, rivers, lakes, houses and other structures, mountains, trees, oceans, cliffs, clouds, and other weather-related constructs. FIG. 3 depicts another preferred embodiment of the virtual environment depicted on the monitor 400, which includes a fairway [300], a plurality of sand traps 310, 312, 314, 316 an ocean 320, mountains 330, and other non-enumerated visual components.

Turning back to FIG. 1, the preferred golf game begins by having the player 300 look at the monitor 400 to determine where they should aim their first golf shot. The virtual environment depicted on the monitor 400 will initially show the virtual golf ball 490 in a virtual tee box of a golf hole (corresponding to the actual golf ball 110 within the actual tee box 100). Depending on the particular shape and length of the fairway of the virtual golf hole depicted within the virtual environment, the player 300 will select a strategy to get his virtual golf ball into the cup on the green of the golf hole using the fewest golf shots. This strategy may involve selecting a particular golf club and striking the golf ball 110 in the tee box 100 thereby causing the golf ball 110 to fly through the air on a particular path onto the range surface 200. That path 495 will be depicted within the virtual environment and shown on the monitor 400. The monitor may then redraw the virtual environment to show the new position of the virtual golf ball 490. Play will continue with the player 300 iteratively targeting a desired physical marker, taking a golf shot, and then watching the monitor draw the path 495 of the virtual golf ball 490 that corresponds to the flight path of the actual golf ball 110 relative to the plurality of physical markers.

It is understood that the various embodiments of the game have different objectives and goals. In the first preferred embodiment, the objective is to get the virtual golf ball 490 to the cup on the golf green 498 taking the fewest number of golf shots possible. In an alternative embodiment of the game, the player 300 is awarded points in successive rounds of the game based upon the how close the resting position of the virtual golf ball 490 is to virtual concentric rings depicted within the virtual environment. In yet another preferred embodiment, the player's goal is to acquire precisely 21 points in each round by causing the path 495 of the virtual golf ball 490 to intercept virtual playing cards, wherein each playing card has a particular value.

It is to be understood that even though numerous characteristics and advantages of various embodiments of the present invention have been set forth in the foregoing description, together with details of the structure and functions of various embodiments of the invention, this disclosure is illustrative only, and changes may be made in detail, especially in matters of structure and arrangement of parts within the principles of the present invention to the full extent indicated by the broad general meaning of the terms in which the appended claims are expressed. It will be appreciated by those skilled in the art that the teachings of the present invention can be applied to other systems without departing from the scope and spirit of the present invention.

What is claimed is:

1. A game-play environment comprising:
a tee box having a monitor; and
a range surface extending from the tee box and having a plurality of physical markers;
wherein the monitor depicts a visual cue that corresponds to each of the plurality of physical markers.

2. The game-play environment of claim 1 further comprising:
a golf ball, wherein the monitor depicts the final resting play of the golf ball after it travels from the tee box through the air and onto the range surface.

3. The game-play environment of claim 2, wherein the monitor depicts the flight path of the golf ball as it travels from the tee box through the air and onto the range surface.

4. The game-play environment of claim 3 wherein the monitor depicts a plurality of visual components that do not correspond to the physical markers.

5. The game-play environment of claim 4 wherein at least one of the visual components is a virtual golf hole.

6. The game-play environment of claim 4 wherein at least one of the visual components is a playing card.

7. The game-play environment of claim 4 wherein at least one of the visual components is a concentric ring.

8. A method of playing a game using a game-play environment, the method comprising the steps of:
targeting a visual component on a monitor;
comparing the targeted visual component to a visual cue that corresponds to a physical marker on a range surface;
aiming the golf shot at the physical marker;
hitting the golf shot and thereby launching a golf ball through the air onto the range surface.

9. The method of claim 8, further comprising the steps of:
tracking a path of the golf ball as it travels through the air onto the range surface; and
depicting the path of the golf ball on the monitor.

10. The method of claim 9 further comprising the step of calculating a score based upon a set of rules, wherein the set of rules provides that a player scores points by landing the golf ball within a distance from the physical marker.

11. The method of claim 9 wherein the visual component is a cup on a green of a golf hole and wherein the set of rules provide that the player scores points by counting the number of golf shots a player takes on the golf hole.

12. The method of claim 9 wherein the set of rules provide that a player scores points when the path of the golf ball depicted on the monitor intersects the visual component.

13. The method of claim 10 wherein the game-play environment is adjusted so that the targeted visual component is depicted in closer proximity with a visual component that does not correspond to a physical marker.

14. A virtual game-play environment comprising:
a first virtual component that corresponds to a first physical marker on a physical game-play surface;
a second virtual component that corresponds to a second physical marker on the physical game-play surface;
wherein the position and direction of the first virtual component relative to the second virtual component correspond to the position and direction of the first physical marker to the second physical marker.

15. The virtual game-play environment of claim 14 further comprising a third virtual component that does not correspond to any physical marker.

16. The virtual game-play environment of claim 15 wherein the third virtual component is a cup on a golf hole.

17. The virtual game-play environment of claim 15 wherein the third virtual component is a directional indicator.

18. The virtual game-play environment of claim 15 wherein the position of the third virtual component can be adjusted relative to the first virtual component and the second virtual component.
